(19) United States
(12) Patent Application Publication
(10) Pub. No.: US 2023/0105000 A1
(43) Pub. Date: Apr. 6, 2023

(54) ELECTROCHEMICAL DEVICE

(71) Applicant: Murata Manufacturing Co., Ltd., Nagaokakyo-shi (JP)

(72) Inventors: Hideaki OOE, Nagaokakyo-shi (JP); Hiroki MITA, Nagaokakyo-shi (JP)

(21) Appl. No.: 17/972,621

(22) Filed: Oct. 25, 2022

Related U.S. Application Data
(63) Continuation of application No. PCT/JP2021/023057, filed on Jun. 17, 2021.

(30) Foreign Application Priority Data
Jun. 23, 2020 (JP) 2020-108054

(51) Int. Cl. H01M 10/0567; H01M 10/0525; C07F 3/06; C07F 15/02; C07F 15/06; C07F 1/08; H01G 11/64

(52) U.S. Cl. CPC H01M 10/0567; H01M 10/0525; C07F 3/06; C07F 15/025; C07F 15/065; C07F 1/08; H01G 11/64; H01M 2300/0025

(57) ABSTRACT

An electrochemical device capable of more sufficiently preventing swelling due to generation of a gas such as carbon dioxide and decomposition of a lithium salt while having a simple structure. The electrochemical device includes a non-aqueous electrolytic solution, wherein the non-aqueous electrolytic solution contains a metal-organic framework containing: an azole-based organic molecule optionally having a hydrophobic group, and a metal atom.

### CROSS REFERENCE TO RELATED APPLICATIONS

The present application is a continuation of International application No. PCT/JP2021/023057, filed Jun. 17, 2021, which claims priority to Japanese Patent Application No. 2020-108054, filed Jun. 23, 2020, the entire contents of each of which are incorporated herein by reference.

### FIELD OF THE INVENTION

The present invention relates to an electrochemical device such as a lithium ion secondary battery and an electric double-layer capacitor.

### BACKGROUND OF THE INVENTION

In the related art, electrochemical devices such as a lithium ion secondary battery and an electric double-layer capacitor have a structure in which a positive electrode, a negative electrode, a separator, and a non-aqueous electrolytic solution are sealed in an exterior body. In such an electrochemical device, the non-aqueous electrolytic solution is oxidized during use, and a gas such as carbon dioxide is generated. Therefore, it is known that safety and reliability are problematic, for example, internal pressure is increased to cause swelling, and in some cases, rupture occurs.

Therefore, in Patent Document 1, an attempt is made to dispose zeolite as a suctioning material in an airtight container separately from an electrolytic solution in order to prevent swelling of a lithium ion battery and improve safety.

On the other hand, in Patent Document 2, attempts have been made to control a melting point of an ionic liquid depending on the application by retaining an ionic liquid in pores of a metal-organic framework (MOF).

Patent Document 3 attempts to improve the life and safety of a lithium secondary battery by using a metal-organic framework (MOF) having a polymerizable reactive group as a polymerization initiator of an electrolyte of the lithium secondary battery.
Patent Document 1: Japanese Patent Application Laid-Open No. 2015-5496
Patent Document 2: WO2013/161452A1
Patent Document 3: Japanese Patent Application Laid-Open No. 2016-62895

### SUMMARY OF THE INVENTION

However, the inventors of the present invention have found that the following new problems arise in a technique in the related art.

In the technique disclosed in Patent Document 1, since porous materials such as zeolite generally have high water absorbability, suction of water at the time of production or before the production cannot be avoided, and when mixed with an electrolytic solution, a Li salt is decomposed to deteriorate characteristics. Therefore, the battery is disposed separately from the electrolytic solution, but the size of the battery is increased, the structure becomes complicated, and the cost is high.

In the technique disclosed in Patent Document 2, although the MOF is contained in the electrolytic solution, since the electrolytic solution exists in the pores of the MOF, there is no gas suction effect. The effect of preventing swelling cannot be obtained.

In the technique disclosed in Patent Document 3, since MOF is used as a polymerization initiator of an electrolyte, a porous structure is not maintained, and a gas suction effect is small. MOF having a polymerizable reactive group deteriorates performance of a battery due to decomposition of a lithium salt because of water absorbability thereof.

An object of the present invention is to provide an electrochemical device capable of more sufficiently preventing swelling due to generation of a gas such as carbon dioxide and decomposition of a lithium salt while having a simple structure.

The present invention relates to an electrochemical device including a non-aqueous electrolytic solution, wherein the non-aqueous electrolytic solution contains a metal-organic framework containing: an azole-based organic molecule optionally having a hydrophobic group, and a metal atom.

In the electrochemical device, it is possible to more sufficiently prevent swelling due to generation of a gas such as carbon dioxide and decomposition of a lithium salt while having a simple structure. Specifically, in the electrochemical device of the present invention, since the metal-organic framework contained in an electrolytic solution has low water absorbability, prevention of swelling can be realized with a simple structure without causing decomposition of the lithium salt even though the metal-organic framework is mixed with the electrolytic solution. More specifically, the gas generated from the electrochemical device can be suctioned without being affected by the electrolytic solution, and the electrochemical device with high safety and reliability can be realized with a simple structure.

### BRIEF EXPLANATION OF THE DRAWINGS

FIG. 1 is a schematic view of a metal-organic framework schematically illustrating a crystal structure of a metal-organic framework contained in a non-aqueous electrolytic solution of an electrochemical device of the present invention.

FIG. 2 is a schematic sectional view of a secondary battery as an example of the electrochemical device of the present invention.

FIG. 3 is a schematic sectional view of a capacitor as an example of the electrochemical device of the present invention.

FIG. 4 is a schematic view for explaining a method for measuring a suction amount of carbon dioxide measured in examples.

### DETAILED DESCRIPTION OF THE INVENTION

[Electrochemical Device]

The electrochemical device of the present invention may be any device utilizing an electrochemical reaction, and generally includes a non-aqueous electrolytic solution. Specific examples of such an electrochemical device include a secondary battery (particularly, a lithium ion secondary battery), a capacitor (particularly, an electric double-layer capacitor), and the like. The non-aqueous electrolytic solution means an electrolytic solution in which a medium in which electrolyte ions move does not contain water, that is, an electrolytic solution using only an organic solvent as a medium. The “secondary battery” is not excessively limited by the name, and may include, for example, a power storage device and the like.

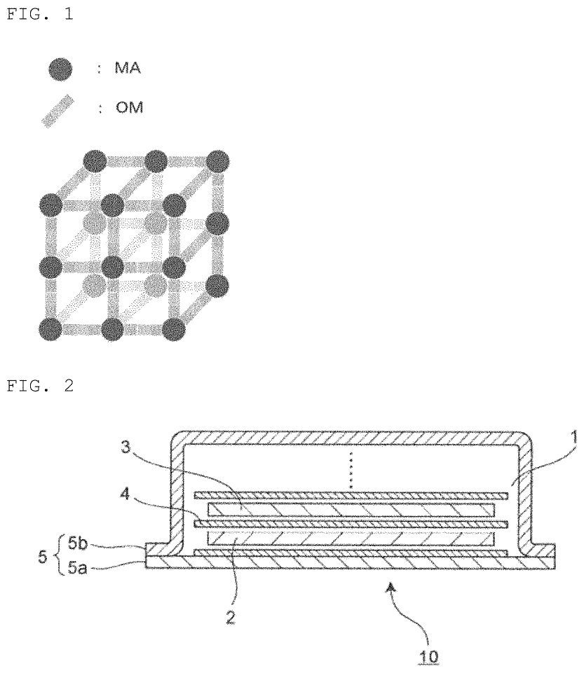

The non-aqueous electrolytic solution contained in the electrochemical device of the present invention contains a specific metal-organic framework (that is, MOF: Metal-Organic Framework). The metal-organic framework is, for example, as illustrated in FIG. 1, a crystalline complex formed by crosslinking a metal atom (particularly, a metal atom ion) MA as a ligand with an organic molecule OM, and is a porous body based on a coordination bond between the organic molecule and the metal atom (particularly, a metal atom ion). In the present specification, various elements in the drawings are merely schematically and exemplarily illustrated for the understanding of the present invention, and appearances, dimensional ratios, and the like may be different from actual ones. Unless otherwise specified, the “vertical direction”, “horizontal direction”, and “front and back direction” used directly or indirectly in the present specification correspond to a vertical direction, a horizontal direction, and a front and back direction in the drawings, respectively. Unless otherwise specified, except that the shape is different, the same reference numerals or symbols indicate the same members or the same semantic contents.

In the present invention, the metal-organic framework contained in the non-aqueous electrolytic solution is a metal-organic framework containing an azole-based organic molecule optionally having a hydrophobic group and a metal atom. What is important here is that the organic molecule constituting the metal-organic framework is an azole-based organic molecule having no substituent, or an azole-based organic molecule having only a hydrophobic group as a substituent although it has a substituent. The azole-based organic molecule as an organic molecule constituting the metal-organic framework does not have a water-absorbent group (or a hydrophilic group) such as an amino group, an imino group, a carboxyl group, a carboxylate group (that is, a carboxylate ester group), a hydroxyl group, a ketone group, or an aldehyde group. Such a metal-organic framework containing an azole-based organic molecule can have suctioning properties of a gas (particularly, carbon dioxide) generated from an electrochemical device while having water absorption resistance. Therefore, it is possible to suction the gas (particularly, carbon dioxide) generated from the electrochemical device and more sufficiently prevent swelling while more sufficiently preventing decomposition of the lithium salt. Specifically, the gas (particularly, carbon dioxide) generated from the electrochemical device can be suctioned on the basis of the porosity of the metal-organic framework, so that the effect of preventing swelling is obtained. At the same time, since the metal-organic framework has water absorption resistance, the effect of preventing swelling can be realized with a simple structure without causing decomposition of a salt although the metal-organic framework is mixed with an electrolytic solution. As a result, an electrochemical device with high safety and reliability can be realized with a simple structure. For example, a porous body such as zeolite is likely to suction water in addition to carbon dioxide gas. Therefore, when the non-aqueous electrolytic solution contains a porous body such as zeolite instead of the metal-organic framework, the lithium salt is decomposed by the reaction with the adsorbed water, hydrofluoric acid is generated, and members such as electrodes are deteriorated. Therefore, reliability as an electrochemical device such as a lithium ion battery or an electric double-layer capacitor is deteriorated. In addition, for example, when the organic molecules constituting the metal-organic framework have a water-absorbent group (or a hydrophilic group), the organic molecules suction water, and thus, as in the case of using a porous body such as zeolite, the lithium salt is decomposed by the reaction with the suctioned water, members such as an electrode are deteriorated, and the reliability as an electrochemical device is deteriorated.

The azole-based organic molecule constituting the metal-organic framework is an organic molecule selected from the group consisting of imidazole, benzimidazole, triazole, and purine. From the viewpoint of further preventing swelling due to gas generation and decomposition of the lithium salt, imidazole, benzimidazole, and purine are preferable, imidazole and benzimidazole are more preferable, and imidazole is still more preferable.

The hydrophobic group optionally contained in the azole-based organic molecule is one or more substituents selected from the group consisting of an alkyl group, a halogen atom, a nitro group, a phenyl group, a pyridyl group, and a cyano group.

The alkyl group is, for example, an alkyl group having 1 to 5 (particularly, 1 to 3) carbon atoms. Specific examples of the alkyl group include a methyl group, an ethyl group, an n-propyl group, an isopropyl group, an n-butyl group, an isobutyl group, a sec-butyl group, a tert-butyl group, and an n-pentyl group.

Examples of the halogen atom include a fluorine atom, a chlorine atom, and a bromine atom.

The hydrophobic group is preferably selected from the group consisting of an alkyl group and a nitro group, and more preferably an alkyl group, from the viewpoint of further preventing swelling due to gas generation and decomposition of the lithium salt.

The azole-based organic molecule constituting the metal-organic framework is preferably an azole-based organic molecule optionally having an alkyl group or a nitro group, more preferably an azole-based organic molecule optionally having an alkyl group, and still more preferably an azole-based organic molecule not having a substituent such as a hydrophobic group or a water-absorbent group, from the viewpoint of further preventing swelling due to gas generation and decomposition of a lithium salt.

Examples of the azole-based organic molecules constituting the metal-organic framework include imidazole-based molecules represented by the following general formula (1), benzimidazole-based molecules represented by the following general formula (2), triazole-based molecules represented by the following general formulas (3) and (4), and purine-based molecules represented by the general formula (5).

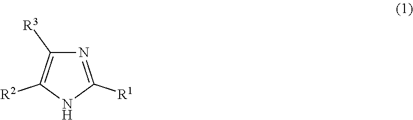

In the formula (1), R¹ to R³ are each independently a hydrogen atom, an alkyl group, a halogen atom, a nitro group, a phenyl group, a pyridyl group, or a cyano group, and are preferably a hydrogen atom, an alkyl group, a halogen atom, a nitro group, or a cyano group from the viewpoint of further preventing swelling due to gas generation and decomposition of the lithium salt. In a more preferable embodiment from the same viewpoint, R¹ is a hydrogen atom, an alkyl group, or a nitro group, and R² and R³ are each independently a hydrogen atom, an alkyl group, a nitro group, a halogen atom, or a cyano group.

Specific examples of the imidazole-based molecule represented by the general formula (1) include the following compounds.

TABLE A

| Compounds | R¹ | R² | R³ |
|---|---|---|---|
| Compound (1-1) | Hydrogen atom | Hydrogen atom | Hydrogen atom |
| Compound (1-2) | Methyl group | Hydrogen atom | Hydrogen atom |
| Compound (1-3) | Ethyl group | Hydrogen atom | Hydrogen atom |
| Compound (1-4) | Nitro group | Hydrogen atom | Hydrogen atom |
| Compound (1-5) | Hydrogen atom | Hydrogen atom | Cyano group |
| Compound (1-6) | Hydrogen atom | Chlorine atom | Chlorine atom |

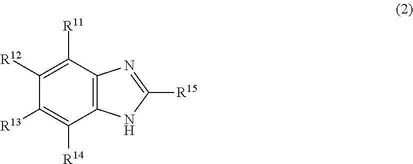

In the formula (2), R¹¹ to R¹⁵ are each independently a hydrogen atom, an alkyl group, a halogen atom, a nitro group, a phenyl group, a pyridyl group, or a cyano group, and are preferably a hydrogen atom, an alkyl group, a halogen atom, or a nitro group from the viewpoint of further preventing swelling due to gas generation and decomposition of the lithium salt. In a more preferable embodiment from the same viewpoint, R¹¹, R¹⁴, and R¹⁵ are a hydrogen atom, and R¹² and R¹³ are each independently a hydrogen atom, an alkyl group, a halogen atom, or a nitro group.

Specific examples of the benzimidazole-based molecule represented by the general formula (2) include the following compounds.

TABLE B

| Compounds | R¹¹ | R¹² | R¹³ | R¹⁴ | R¹⁵ |
|---|---|---|---|---|---|
| Compound (2-1) | Hydrogen atom | Hydrogen atom | Hydrogen atom | Hydrogen atom | Hydrogen atom |
| Compound (2-2) | Hydrogen atom | Chlorine atom | Hydrogen atom | Hydrogen atom | Hydrogen atom |
| Compound (2-3) | Hydrogen atom | Bromine atom | Hydrogen atom | Hydrogen atom | Hydrogen atom |
| Compound (2-4) | Hydrogen atom | Methyl group | Hydrogen atom | Hydrogen atom | Hydrogen atom |
| Compound (2-5) | Hydrogen atom | Methyl group | Methyl group | Hydrogen atom | Hydrogen atom |
| Compound (2-6) | Hydrogen atom | Nitro group | Hydrogen atom | Hydrogen atom | Hydrogen atom |

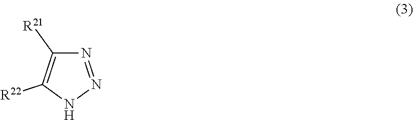

In the formula (3), R²¹ to R²² are each independently a hydrogen atom, an alkyl group, a halogen atom, a nitro group, a phenyl group, a pyridyl group, or a cyano group, and are preferably a hydrogen atom, an alkyl group, a halogen atom, a phenyl group, or a nitro group from the viewpoint of further preventing swelling due to gas generation and decomposition of the lithium salt. In a more preferable embodiment from the same viewpoint, R²¹ is a hydrogen atom, a halogen atom, or a nitro group, and R²² is a hydrogen atom, a halogen atom, or a nitro group.

Specific examples of the triazole-based molecule represented by the general formula (3) include the following compounds.

TABLE C

| Compounds | R²¹ | R²² |
|---|---|---|
| Compound (3-1) | Hydrogen atom | Hydrogen atom |
| Compound (3-2) | Hydrogen atom | Nitro group |
| Compound (3-3) | Nitro group | Hydrogen atom |
| Compound (3-4) | Bromine atom | Bromine atom |
| Compound (3-5) | Hydrogen atom | Chlorine atom |

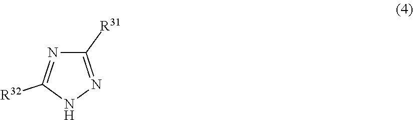

In the formula (4), R³¹ to R³² are each independently a hydrogen atom, an alkyl group, a halogen atom, a nitro group, a phenyl group, a pyridyl group, or a cyano group, and are preferably a hydrogen atom, an alkyl group, a halogen group, a nitro group, or a phenyl group from the viewpoint of further preventing swelling due to gas generation and decomposition of the lithium salt. In a more preferable embodiment from the same viewpoint, R³¹ is a hydrogen atom, a halogen atom, or a nitro group, and R³² is a hydrogen atom, a halogen atom, or a nitro group.

Specific examples of the triazole-based molecule represented by the general formula (4) include the following compounds.

TABLE D

| Compounds | R³¹ | R³² |
|---|---|---|
| Compound (4-1) | Hydrogen atom | Hydrogen atom |
| Compound (4-2) | Hydrogen atom | Nitro group |
| Compound (4-3) | Hydrogen atom | Methyl group |
| Compound (4-4) | Hydrogen atom | Bromine atom |
| Compound (4-5) | Hydrogen atom | Chlorine atom |
| Compound (4-6) | Bromine atom | Bromine atom |

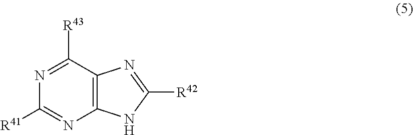

In the formula (5), R⁴¹ to R⁴³ are each independently a hydrogen atom, an alkyl group, a halogen atom, a nitro group, a phenyl group, a pyridyl group, or a cyano group, and are preferably a hydrogen atom, an alkyl group, a halogen atom, a nitro group, or a phenyl group from the viewpoint of further preventing swelling due to gas generation and decomposition of the lithium salt. In a more preferable embodiment from the same viewpoint, R⁴¹ is a hydrogen atom or a halogen atom, R⁴² is a hydrogen atom or a nitro group, and R⁴³ is a hydrogen atom or halogen atom, or a nitro group.

Specific examples of the purine-based molecule represented by the general formula (5) include the following compounds.

TABLE E

| Compounds | R⁴¹ | R⁴² | R⁴³ |
|---|---|---|---|
| Compound (5-1) | Hydrogen atom | Hydrogen atom | Hydrogen atom |
| Compound (5-2) | Hydrogen atom | Nitro group | Hydrogen atom |
| Compound (5-3) | Bromine atom | Hydrogen atom | Hydrogen atom |
| Compound (5-4) | Bromine atom | Hydrogen atom | Bromine atom |
| Compound (5-5) | Hydrogen atom | Hydrogen atom | Bromine atom |
| Compound (5-6) | Chlorine atom | Hydrogen atom | Hydrogen atom |

The metal atom constituting the metal-organic framework is selected from the group consisting of a zinc atom, a cobalt atom, an iron atom, a praseodymium atom, a cadmium atom, a mercury atom, a copper atom, an indium atom, a manganese atom, a lithium atom, and a boron atom, and is preferably selected from the group consisting of a zinc atom, a cobalt atom, and an iron atom, more preferably selected from the group consisting of a zinc atom and a cobalt atom, and still more preferably a zinc atom, from the viewpoint of further preventing swelling due to gas generation and decomposition of a lithium salt.

The combination of the organic molecule and the metal atom in the metal-organic framework is not particularly limited, and from the viewpoint of further preventing swelling due to gas generation and decomposition of the lithium salt, preferably the following combination:

Combination (C1)=a combination of an imidazole-based molecule represented by general formula (1) and one or more metal atoms selected from the group consisting of a zinc atom and an iron atom;

Combination (C2)=a combination of a benzimidazole-based molecule represented by general formula (2) and one or more metal atoms selected from the group consisting of a zinc atom and a cobalt atom;

Combination (C3)=a combination of a triazole-based molecule represented by general formula (3) and one or more metal atoms selected from the group consisting of a zinc atom and a cobalt atom;

Combination (C4)=a combination of a triazole-based molecule represented by general formula (4) and one or more metal atoms selected from the group consisting of a zinc atom and a cobalt atom;

Combination (C5)=combination of purine-based molecule represented by general formula (5) and one or more metal atoms selected from the group consisting of a zinc atom and a cobalt atom;

Combination (C6)=combination of an imidazole-based molecule represented by general formula (1), a benzimidazole-based molecule represented by general formula (2), and a cobalt atom.

A ratio between the organic molecule and the metal atom in the metal-organic framework is not particularly limited, and is generally determined by the type of the organic molecule and the type of the metal atom constituting the metal-organic framework.

For example, a metal-organic framework containing an imidazole-based molecule (Im) (for example, an imidazole-based molecule represented by the general formula (1)) and one or more metal atoms (M¹) selected from the group consisting of a zinc atom, a cobalt atom, an iron atom, a copper atom, a manganese atom, an indium atom, a cadmium atom, a lithium atom, and a boron atom can be represented by the composition formula: M¹ (Im)₂; here, the boron atom is not necessarily classified into a metal in some cases, but is described as a metal atom here since the metal-organic frameworks have properties similar to those of metals (the same applies hereafter).

In addition, for example, a metal-organic framework containing a benzimidazole-based molecule (bIm) (for example, a benzimidazole-based molecule represented by the general formula (2)) and one or more metal atoms (M¹) selected from the group consisting of a zinc atom, a cobalt atom, an iron atom, a copper atom, a manganese atom, an indium atom, a cadmium atom, a lithium atom, and a boron atom can be represented by the composition formula: M¹ (bIm)₂.

In addition, for example, a metal-organic framework containing a triazole-based molecule (Tra) (for example, a triazole-based molecule represented by the general formula (3) and/or (4)) and one or more metal atoms (M¹) selected from the group consisting of a zinc atom, a cobalt atom, an iron atom, a copper atom, a manganese atom, an indium atom, a cadmium atom, a lithium atom, and a boron atom can be represented by the composition formula: M¹(Tra)₂.

In addition, for example, the metal-organic framework containing a purine-based molecule (Pur) (for example, a triazole-based molecule represented by the general formula (5)) and one or more metal atoms (M¹) selected from the group consisting of a zinc atom, a cobalt atom, an iron atom, a copper atom, a manganese atom, an indium atom, a cadmium atom, a lithium atom, and a boron atom can be represented by the composition formula: M¹(Pur)₂.

For example, a metal-organic framework containing an imidazole-based molecule (Im) (for example, an imidazole-based molecule represented by the general formula (1)), a benzimidazole-based molecule (bIm) (for example, a benzimidazole-based molecule represented by the general formula (2)), and one or more metal atoms (M¹) selected from the group consisting of a zinc atom, a cobalt atom, an iron atom, a copper atom, a manganese atom, an indium atom, a cadmium atom, a lithium atom, and a boron atom can be represented by the composition formula: M¹(Im)ₓ(bIm)_{y} (wherein x+y=2).

In addition, for example, a metal-organic framework containing an imidazole-based molecule (Im) (for example, an imidazole-based molecule represented by the general formula (1)) and two or more metal atoms (M¹ and M²) selected from the group consisting of a zinc atom, a cobalt atom, an iron atom, a copper atom, a manganese atom, an indium atom, a cadmium atom, a lithium atom, and a boron atom can be represented by the composition formula: M¹M²(Im)₄.

The metal-organic framework can be synthesized by mixing a compound containing a predetermined organic molecule and a predetermined metal atom in an aqueous solvent or an organic solvent. It can be produced by heating to 60° C. to 150° C. in order to promote grain growth. Examples of the compound containing a predetermined metal atom include zinc nitrate, cobalt nitrate, and iron nitrate. Examples of the organic solvent include N,N-diethylformamide, N,N-dimethylformamide, and methanol.

The metal-organic framework is also available as commercial products.

For example, for the combination (C1) described above, a metal-organic framework containing a combination of 2-methylimidazole and a zinc atom is available as commercially available ZIF-8 (Product name: BASOLITE 21200 available from Sigma Aldrich, composition formula: Zn (mIm)₂).

In the present invention, the metal-organic framework contained in the non-aqueous electrolytic solution generally has a pore diameter of 1 Å to 50 Å, and from the viewpoint of further preventing swelling due to gas generation and decomposition of the lithium salt, the metal-organic framework preferably has a pore diameter of 1 Å to 15 Å, particularly preferably 1.5 Å to 5 Å, more preferably 2 Å to 5 Å, and still more preferably 2 Å to 3 Å. Although a dynamic molecular diameter of carbon dioxide is 3.3 Å, in the present invention, carbon dioxide can be suctioned or captured although the pore diameter of the metal-organic framework is less than 3.3 Å. Moreover, carbon dioxide suctioned or captured by such a metal-organic framework is hardly liberated. This is considered to be due to the flexibility of organic molecules contained in the metal-organic frameworks as ligands. As described above, the fact that carbon dioxide is captured and hardly released although the pore diameter is relatively small is considered to further contribute to prevention of swelling due to gas generation and decomposition of the lithium salt.

The pore diameter depends on the types (particularly, bulkiness and size) of organic molecules and metal atoms constituting the metal-organic framework. Therefore, the pore diameter can be adjusted by selecting the types of organic molecules and metal atoms.

In the present specification, the pore diameter is defined as “a diameter of the largest sphere in which each atom in a crystal can be included when the atom is assumed to be a rigid sphere with a van der Waals radius”, and is a pore diameter in a state where no molecule is contained in the pore. Therefore, the pore diameter can be calculated from the crystal structure. Such a pore diameter is described as dₚ (A) in Table 1 of the following document, and the value described in this document can be used: ANH PHAN et al., “Synthesis, Structure, and Carbon Dioxide Capture Properties of Zeolitic Imidazolate Frameworks” (ACCOUNTS OF CHEMICAL RESEARCH 58 67 January 2010 Vol. 43, No. 1)

The metal-organic framework generally has an average particle diameter of 0.01 μm to 1 μm in the non-aqueous electrolytic solution, and preferably has an average particle diameter of 0.02 μm to 0.5 μm, and more preferably 0.05 μm to 0.2 μm from the viewpoint of further preventing swelling due to gas generation and decomposition of the lithium salt.

As the average particle diameter of the metal-organic framework, an average value related to the maximum length of optional 100 metal-organic framework particles based on a micrograph is used.

The content of the metal-organic framework is not particularly limited, and is generally 0.1 wt. % to 50 wt. % with respect to the total amount of the non-aqueous electrolytic solution, and is preferably 1 wt. % to 5 wt. % from the viewpoint of further preventing swelling due to gas generation and decomposition of the lithium salt. The non-aqueous electrolytic solution may contain two or more types of metal-organic frameworks in which the structures of organic molecules and/or the types of metal atoms are different from each other, and in that case, the total content thereof may be within the above range.

The non-aqueous electrolytic solution generally further contains an organic solvent and an electrolyte salt in addition to the metal-organic framework.

Examples of the organic solvent include all organic solvents known in the related art in the field of the non-aqueous electrolytic solution of the electrochemical device. Specific examples of the organic solvent include cyclic carbonates of γ1-butyrolactone such as propylene carbonate (PC), ethylene carbonate (EC), butylene carbonate (BC), and vinylene carbonate (VC); chain carbonates such as dimethyl carbonate (DMC), diethyl carbonate (DEC), ethyl methyl carbonate (EMC), dipropyl carbonate (DPC), and methylethyl carbonate; tetrahydrofuran, dioxolane, sulfolane, dimethylformamide, dimethylacetamide, and N-methyl-2-pyrrolidone.

The content of the organic solvent is generally 40 wt. % to 95 wt. % with respect to the total amount of the non-aqueous electrolytic solution, and is preferably 70 wt. % to 90 wt. % from the viewpoint of further preventing swelling due to gas generation and decomposition of the lithium salt.

Examples of the electrolyte salt include all electrolyte salts known in the related art in the field of the non-aqueous electrolytic solution of the electrochemical device. Specific examples of the electrolyte salt include LiPF₆, LiBF₄, LiClO₄, LiCF₃SO₃, LiN(CF₃SO₂)₂, LiN (C₂F₅SO₂)₂, LiC(CF₃SO₂)₃, and LiC(C₂F₅SO₂)₃.

The content of the electrolyte salt is generally 5 wt. % to 25 wt. % with respect to the total amount of the non-aqueous electrolytic solution, and is preferably 10 wt. % to 20 wt. % from the viewpoint of further preventing swelling due to gas generation and decomposition of the lithium salt.

The non-aqueous electrolyte may further contain any additive known in the related art in the field of the non-aqueous electrolytic solution for the electrochemical device (for example, a binder, a filler, or the like).

Examples of the binder include polyacrylonitrile, polyvinylidene fluoride, polytetrafluoroethylene, polyhexafluoropropylene, polyethylene oxide, polypropylene oxide, polyphosphazene, polysiloxane, polyvinyl fluoride (PVF), polychlorotrifluoroethylene (PCTFE), perfluoroalkoxy fluorine resin (PFA), an ethylene tetrafluoride-propylene hexafluoride copolymer (FEP), an ethylene-ethylene tetrafluoride copolymer (ETFE), an ethylene-chlorotrifluoroethylene copolymer (ECTFE), polyvinyl acetate, polyvinyl alcohol, polymethyl methacrylate, polyacrylic acid, polymethacrylic acid, styrene-butadiene rubber, nitrile-butadiene rubber, polystyrene, polycarbonate, polyethylene oxide, and vinyl chloride. The binder may be used singly or in combination of two or more kinds thereof. The binder may be a copolymer composed of two or more monomers constituting the binder. Specific examples of the copolymer include a copolymer of vinylidene fluoride and hexafluoropyrene. Among them, polyvinylidene fluoride and a copolymer of vinylidene fluoride and hexafluoropyrene are preferable from the viewpoint of electrochemical stability.

As the filler, a compound having high heat resistance such as Al₂O₃, SiO₂, TiO₂, and BN (boron nitride) may be contained.

The non-aqueous electrolytic solution can be obtained by mixing a metal-organic framework, an organic solvent, an electrolyte salt, and other desired additives. The non-aqueous electrolytic solution may have a form such as a liquid form or a gel form.

[Secondary Battery]

When the electrochemical device of the present invention is a secondary battery, in the secondary battery, a positive electrode, a negative electrode, a separator, and the like are sealed in an exterior body in addition to the above-described non-aqueous electrolytic solution. In a plan view, a seal portion (sealing portion) for holding a non-aqueous electrolytic solution or the like is generally formed in an exterior body at a peripheral edge portion of the secondary battery. The plan view is a state when the secondary battery is placed and viewed from directly above in the thickness (height) direction, and is the same as a ground plan. The placement is, for example, placement in which the surface having the maximum area of the secondary battery is a bottom surface. In the present specification, the term “secondary battery” refers to a battery that can be repeatedly charged and discharged.

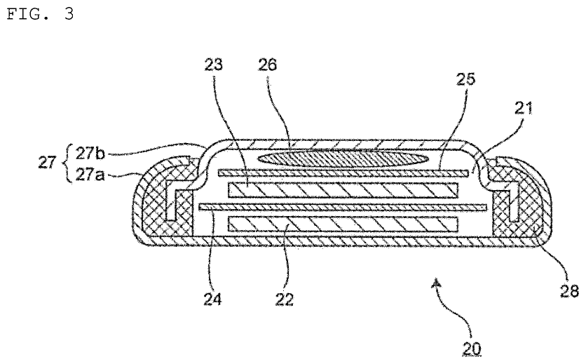

As illustrated in FIG. 2, for example, a secondary battery 10 of the present invention includes a non-aqueous electrolytic solution 1, a positive electrode 2, a negative electrode 3, and a separator 4, and the positive electrode 2 and the negative electrode 3 are alternately arranged with the separator 4 interposed therebetween. The two external terminals (not shown) are connected to the electrode (positive electrode or negative electrode) via a current collecting lead (not shown), and as a result, are led out from the seal portion to the outside. The non-aqueous electrolytic solution 1 assists movement of metal ions released from the electrodes (positive electrode and negative electrode). In FIG. 2, the secondary battery 10 has a planar laminated structure in which the positive electrode 2, the negative electrode 3, and the separator 4 disposed between the positive electrode 2 and the negative electrode 3 are laminated in a planar shape, but is not limited to the planar laminated structure. For example, the secondary battery may have a wound structure in which the positive electrode 2, the negative electrode 3, and the separator 4 disposed between the positive electrode 2 and the negative electrode 3 is wound in a roll shape. For example, the secondary battery may have a so-called stack-and-folding structure in which the positive electrode 2, the negative electrode 3, and the separator 4 disposed between the positive electrode 2 and the negative electrode 3 are laminated and then folded. FIG. 2 is a schematic sectional view of a secondary battery as an example of the electrochemical device of the present invention.

The positive electrode 2 is generally composed of at least a positive electrode layer and a positive electrode current collector (foil), and the positive electrode layer is provided on at least one side of the positive electrode current collector. For example, in the positive electrode 2, the positive electrode layer may be provided on both surfaces of the positive electrode current collector, or the positive electrode layer may be provided on one surface of the positive electrode current collector. In the positive electrode 2 which is preferable from the viewpoint of further increasing the capacity of the secondary battery, the positive electrode layers are provided on both surfaces of a positive electrode current collector. The positive electrode layer contains a positive electrode active material.

The negative electrode 3 is generally composed of at least a negative electrode layer and a negative electrode current collector (foil), and the negative electrode layer is provided on at least one side of the negative electrode current collector. For example, in the negative electrode 3, the negative electrode layer may be provided on both surfaces of the negative electrode current collector, or the negative electrode layer may be provided on one surface of the negative electrode current collector. In the negative electrode 3 which is preferable from the viewpoint of further increasing the capacity of the secondary battery, the negative electrode layers are provided on both surfaces of the negative electrode current collector. The negative electrode layer contains a negative electrode active material.

The positive electrode active material contained in the positive electrode layer and the negative electrode active material contained in the negative electrode layer are substances directly involved in the transfer of electrons in the secondary battery, and are main substances of the positive and negative electrodes responsible for charge and discharge, that is, a battery reaction. More specifically, ions are brought in the non-aqueous electrolytic solution due to the “positive electrode active material contained in the positive electrode layer” and the “negative electrode active material contained in the negative electrode layer”, and such ions move between the positive electrode and the negative electrode to transfer electrons, and charging and discharging are performed. The positive electrode layer and the negative electrode layer are particularly preferably layers capable of occluding and releasing lithium ions. That is, a secondary battery in which lithium ions move between the positive electrode and the negative electrode through the non-aqueous electrolytic solution to charge and discharge the battery is preferable. When the lithium ions are involved in charging and discharging, the secondary battery according to the present embodiment corresponds to a so-called “lithium ion secondary battery”.

The positive electrode active material of the positive electrode layer is made of, for example, a granular material, and it is preferable that a binder is contained in the positive electrode layer for sufficient contact between particles and shape retention. Furthermore, it is also preferable that a conductive auxiliary agent is contained in the positive electrode layer in order to facilitate transfer of electrons promoting the battery reaction. Similarly, when the negative electrode active material of the negative electrode layer is made of, for example, a granular material, a binder is preferably contained for sufficient contact between particles and shape retention, and a conductive auxiliary agent may be contained in the negative electrode layer in order to facilitate transmission of electrons promoting a battery reaction. As described above, since a plurality of components are contained, the positive electrode layer and the negative electrode layer can also be referred to as a “positive electrode mixture layer” and a “negative electrode mixture layer”, respectively.

The positive electrode active material is preferably a material that contributes to occlusion and release of lithium ions. From such a viewpoint, the positive electrode active material is preferably, for example, a lithium-containing composite oxide. More specifically, the positive electrode active material is preferably a lithium transition metal composite oxide containing lithium and at least one transition metal selected from the group consisting of cobalt, nickel, manganese, and iron. That is, in the positive electrode layer of the secondary battery according to the present embodiment, such a lithium transition metal composite oxide is preferably contained as a positive electrode active material. For example, the positive electrode active material may be lithium cobaltate, lithium nickelate, lithium manganate, lithium titanate, or a material obtained by replacing a part of these transition metals with another metal. Such a positive electrode active material may be contained as a single type, but two or more types may be contained in combination. In a more preferable embodiment, the positive electrode active material contained in the positive electrode layer is lithium cobalt oxide.

The binder that can be contained in the positive electrode layer is not particularly limited, and examples thereof include at least one selected from the group consisting of polyvinylidene fluoride, a vinylidene fluoride-hexafluoropropylene copolymer, a vinylidene fluoride-tetrafluoroethylene copolymer, and polytetrafluoroethylene. The conductive auxiliary agent that can be contained in the positive electrode layer is not particularly limited, and examples thereof include at least one selected from carbon black such as thermal black, furnace black, channel black, ketjen black, and acetylene black, metal powders such as copper, nickel, aluminum, and silver, and polyphenylene derivatives. In a more preferable embodiment, the binder of the positive electrode layer is polyvinylidene fluoride, and in another more preferable embodiment, the conductive auxiliary agent of the positive electrode layer is carbon black. In a further preferable embodiment, the binder and the conductive auxiliary agent of the positive electrode layer are a combination of polyvinylidene fluoride and carbon black.

The negative electrode active material is preferably a material that contributes to occlusion and release of lithium ions. From such a viewpoint, the negative electrode active material is preferably, for example, various carbon materials, oxides, lithium alloys, or the like.

Examples of various carbon materials of the negative electrode active material include graphite (natural graphite, artificial graphite), hard carbon, and diamond-like carbon. In particular, graphite is preferable because it has high electron conductivity and excellent adhesion to the negative electrode current collector. Examples of the oxide of the negative electrode active material include at least one selected from the group consisting of silicon oxide, tin oxide, indium oxide, zinc oxide, and lithium oxide. The lithium alloy of the negative electrode active material may be any metal that can be alloyed with lithium, and may be, for example, a binary, ternary, or higher alloy of lithium and a metal such as Al, Si, Pb, Sn, In, Bi, Ag, Ba, Ca, Hg, Pd, Pt, Te, Zn, or La. Such an oxide is preferably amorphous as its structural form. This is because deterioration due to nonuniformity such as crystal grain boundaries or defects is less likely to occur. In a more preferable embodiment, the negative electrode active material of the negative electrode layer is artificial graphite.

The binder that can be contained in the negative electrode layer is not particularly limited, and examples thereof include at least one selected from the group consisting of styrene butadiene rubber, polyvinylidene fluoride, a polyimide-based resin, and a polyamideimide-based resin. In a more preferable embodiment, the binder contained in the negative electrode layer is styrene butadiene rubber. The conductive auxiliary agent that can be contained in the negative electrode layer is not particularly limited, and examples thereof include at least one selected from carbon black such as thermal black, furnace black, channel black, ketjen black, and acetylene black, metal powders such as copper, nickel, aluminum, and silver, and polyphenylene derivatives. The negative electrode layer may contain a component derived from a thickener component (for example, carboxymethyl cellulose) used at the time of producing the battery.

In a more preferable embodiment, the negative electrode active material and the binder in the negative electrode layer are a combination of artificial graphite and styrene-butadiene rubber.

The positive electrode current collector and the negative electrode current collector used for the positive electrode and the negative electrode are members that contribute to collecting and supplying electrons generated in the active material due to the battery reaction. Such a current collector may be a sheet-like metal member and may have a porous or perforated form. For example, the current collector may be a metal foil, a punching metal, a net, an expanded metal, or the like. The positive electrode current collector used for the positive electrode is preferably made of a metal foil containing at least one selected from the group consisting of aluminum, stainless steel, nickel, and the like, and may be, for example, an aluminum foil. On the other hand, the negative electrode current collector used for the negative electrode is preferably made of a metal foil containing at least one selected from the group consisting of copper, stainless steel, nickel, and the like, and may be, for example, a copper foil.

The separator 4 is a member provided from the viewpoint of preventing a short circuit due to contact between the positive and negative electrodes, holding the non-aqueous electrolytic solution, and the like. In other words, it can be said that the separator is a member that allows ions to pass while preventing electronic contact between the positive electrode and the negative electrode. Preferably, the separator is a porous or microporous insulating member, and has a membrane form due to its small thickness. Although it is merely an example, a microporous membrane made of polyolefin may be used as the separator. In this regard, the microporous membrane used as the separator may contain, for example, only polyethylene (PE) or a material containing only polyethylene (PP), as polyolefin. Furthermore, the separator may be a laminate composed of a “microporous membrane made of PE” and a “microporous membrane made of PP”.

An exterior body 5 is preferably a flexible pouch (soft bag body), and may be a hard case (hard housing). When the exterior body 5 is a flexible pouch, the flexible pouch is generally formed of a laminate film, and a seal portion is formed by heat-sealing a peripheral edge portion. As the laminate film, a film obtained by laminating a metal foil and a polymer film is generally used, and specifically, a film having a three-layer structure including an outer layer polymer film/metal foil/inner layer polymer film is exemplified. The outer layer polymer film is for preventing damage to the metal foil due to permeation and contact of moisture and the like, and polymers such as polyamide and polyester can be suitably used. The metal foil is for preventing permeation of moisture and gas, and a foil of copper, aluminum, stainless steel, or the like can be suitably used. The inner layer polymer film is for protecting the metal foil from the electrolyte housed inside and for melt-sealing at the time of heat sealing, and polyolefin or acid-modified polyolefin can be suitably used. The thickness of the laminate film is not particularly limited, and is preferably, for example, 1 μm to 1 mm. For example, in the secondary battery 10 illustrated in FIG. 2, the exterior body 5 is a flexible pouch, and a lower film 5a and an upper film 5b are heat-sealed at peripheral edge portions thereof in plan view.

When the exterior body 6 is a hard case, the hard case is generally formed of a metal plate, and a seal portion is formed by irradiating a peripheral edge portion with laser. As the metal plate, a metal material made of aluminum, nickel, iron, copper, stainless steel, or the like is generally used. The thickness of the metal plate is not particularly limited, and is preferably, for example, 1 μm to 1 mm.

The secondary battery can be available from the following method.

First, the positive electrode 2 and the negative electrode 3 are produced. Specifically, the positive electrode 2 can be obtained by mixing a positive electrode active material, a binder, and the like together, adding an organic solvent to prepare a slurry, coating the slurry on a positive electrode current collector by an optional coating method, and drying the slurry. The negative electrode 3 can be obtained by mixing a negative electrode active material, a binder, and the like together, adding an organic solvent to prepare a slurry, coating the slurry on a negative electrode current collector by an optional coating method, and drying the slurry. The organic solvent contained in the slurry for producing the positive electrode and the negative electrode of the secondary battery is not particularly limited, and for example, an organic solvent such as a basic solvent such as dimethyl sulfoxide, dimethylformamide, N-methyl-2-pyrrolidone, propylene carbonate, diethyl carbonate, dimethyl carbonate, or γ-butyrolactone, a nonaqueous solvent such as acetonitrile, tetrahydrofuran, nitrobenzene, or acetone, or a protic solvent such as methanol or ethanol can be used.

Next, a positive electrode lead (not shown) is attached to the positive electrode 2, a negative electrode lead (not shown) is attached to the negative electrode 3, and the positive electrode 2 and the negative electrode 3 are laminated with the separator 4 interposed therebetween to form a laminated electrode body. If necessary, the laminated electrode body is wound to produce a wound electrode body, and then a protective tape is attached to the outermost peripheral portion of the wound electrode body.

The remaining outer peripheral edge portion excluding the outer peripheral edge portion of one side of the outer peripheral edge portion of the exterior body 5 (5a, 5b) in a plan view is bonded using a thermal fusion method or the like to form a bag-shaped exterior body. The laminated electrode body or the wound electrode body is housed therein.

After the non-aqueous electrolytic solution 1 is injected into the bag-shaped exterior body, the exterior body is sealed using a thermal fusion method or the like.

If necessary, a heat treatment for monomer thermal polymerization or the like may be performed.

[Electric Double-Layer Capacitor]

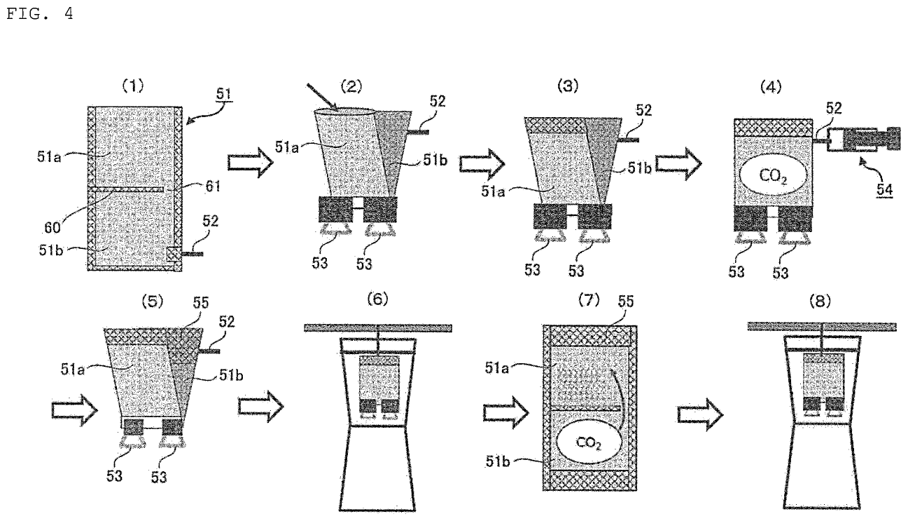

When the electrochemical device of the present invention is an electric double-layer capacitor, in the electric double-layer capacitor, a positive electrode, a negative electrode, a separator, and the like are sealed in an exterior body in addition to the above-described non-aqueous electrolytic solution. As illustrated in FIG. 3, the exterior body 27 includes a positive electrode case 27a and a negative electrode case 27b, and both the positive electrode case 27a and the negative electrode case 27b are formed in a disc-like thin plate shape. A positive electrode 22 containing a positive electrode active material (electrode active material) and a conductive agent is disposed at the bottom center of the positive electrode case 27a. That is, in the positive electrode 22, a mixture containing a positive electrode active material (electrode active material) and a conductive agent is formed into a sheet shape on a positive electrode current collector. A separator 24 formed of a porous sheet or film such as a microporous membrane, a woven fabric, or a nonwoven fabric is laminated on the positive electrode 22, and a negative electrode 23 is further laminated on the separator 24. That is, in the negative electrode 23, similarly to the positive electrode 22, a mixture containing a negative electrode active material (electrode active material) and a conductive agent is formed into a sheet shape on a metal negative electrode current collector 25. The negative electrode 23 is disposed to face the positive electrode 22 with the separator 24 interposed therebetween, and a metallic spring 26 is placed on the negative electrode current collector 25. The internal space is filled with the non-aqueous electrolytic solution 21, and the negative electrode case 27b is fixed to the positive electrode case 27a against the biasing force of the metallic spring 26 and sealed with a gasket 28 interposed therebetween. FIG. 3 is a schematic sectional view schematically illustrating a coin type electric double-layer capacitor as one embodiment of the electric double-layer capacitor according to the present invention.

In the electric double-layer capacitor 20, charged particles in the non-aqueous electrolytic solution 21 are irregularly distributed in the non-aqueous electrolytic solution 21 before a voltage is applied between the positive electrode 22 and the negative electrode 23. On the other hand, when a voltage is applied between the positive electrode 22 and the negative electrode 23, positive ions in the positive electrode 22 and negative ions in the non-aqueous electrolytic solution 21 are distributed in pairs at an interface between the positive electrode 22 (positive electrode active material) and the non-aqueous electrolytic solution 21. In addition, the negative ions in the negative electrode 23 and positive ions in the non-aqueous electrolytic solution 21 are distributed in pairs at an interface between the negative electrode (negative electrode active material) 23 and the non-aqueous electrolytic solution 21. As a result, the positive ions and the negative ions are distributed in layers at the contact interface with the non-aqueous electrolytic solution 21 on the positive electrode 22 side, and negative ions and positive ions are distributed in layers at the contact interface with the non-aqueous electrolytic solution 21 on the negative electrode 23 side, thereby forming an electric double layer having a large surface area.

As the positive electrode active material, any material that can be used as a positive electrode active material in the field of the electric double-layer capacitors can be used. Specific examples of the positive electrode active material include activated carbon.

As the negative electrode active material, any material that can be used as a negative electrode active material in the field of the electric double-layer capacitors can be used. Specific examples of the negative electrode active material include carbon.

The conductive agent that can be contained in the positive electrode and the negative electrode is not particularly limited, and for example, carbonaceous fine particles such as graphite, carbon black, and acetylene black, carbon fibers such as vapor grown carbon fibers, carbon nanotubes, and carbon nanohorns, and conductive polymers such as polyaniline, polypyrrole, polythiophene, polyacetylene, and polyacene can be used. The conductive agent can be used singly or in combination of two or more types thereof.

The positive electrode and the negative electrode may each independently contain a binder. As the binder, any binder that can be used as a binder in the fields of the positive electrode and the negative electrode of the electric double-layer capacitor can be used. Specific examples of such a binder include polyethylene, polypropylene, polyvinylidene fluoride, polyhexafluoropropylene, polytetrafluoroethylene, polyethylene oxide, carboxymethyl cellulose, a styrene-butadiene copolymer, and methyl polyacrylate. The binder can be used singly or in combination of two or more types thereof.

The separator 24 may be selected from the same range as the separator 4 of the secondary battery.

The electric double-layer capacitor can be available from the following method.

First, the positive electrode 22 and the negative electrode 23 are produced. Specifically, the positive electrode 22 can be obtained by mixing a positive electrode active material, a conductive agent, a binder, and the like together, adding an organic solvent to prepare a slurry, coating the slurry on a positive electrode current collector by an optional coating method, and drying the slurry. The negative electrode 23 can be obtained by mixing a negative electrode active material, a conductive agent, a binder, and the like together, adding an organic solvent to prepare a slurry, coating the slurry on a negative electrode current collector by an optional coating method, and drying the slurry. The organic solvent contained in the slurry for producing the positive electrode and the negative electrode of the electric double-layer capacitor is not particularly limited, and for example, an organic solvent similar to the organic solvent contained in the slurry for producing the positive electrode and the negative electrode of the secondary battery may be used.

Next, the positive electrode 22 is impregnated with the non-aqueous electrolytic solution 21, the negative electrode 23 and the negative electrode current collector 25 are disposed so as to face the positive electrode 22 via the separator 24 impregnated with the non-aqueous electrolytic solution 21, and then the non-aqueous electrolytic solution 21 is injected into the internal space. Then, the metallic spring 26 is seated on the negative electrode current collector 25, the gasket 28 is disposed on the peripheral edge, and the negative electrode case 27b is fixed to the positive electrode case 27a by a crimping machine or the like to be externally sealed, thereby preparing a coin-type electric double-layer capacitor.

The electric double-layer capacitor according to the present embodiment has been described as a coin type electric double-layer capacitor, but the shape is not particularly limited. The electric double-layer capacitor may be a cylindrical type, a square type, a sheet type, or the like. The exterior body 27 is also not particularly limited, and a metal case, a mold resin, an aluminum laminate film, or the like may be used.

### EXAMPLES

[Production of Non-Aqueous Electrolytic Solution]

### Example 1

Organic solvent: EC/PC (mass ratio: 1/1)
Lithium salt: 1.0 mol of LiPF₆/1 kg of organic solvent

ZIF-4 (sample) in an amount of 2 wt. % relative to the total amount was mixed with the above-described organic solvent and lithium salt, and the mixture was sufficiently stirred with an ultrasonic homogenizer to obtain a non-aqueous electrolytic solution. In the non-aqueous electrolytic solution, the lithium salt is dissolved, and the sample is dispersed.

### Examples 2 to 6 and Comparative Examples 1 to 4

A non-aqueous electrolytic solution was obtained by the same method as in Example 1 except that the sample was changed to the sample described in tables.

Specifically, the “ZIF-4” used in Example 1 is a substance included in the category of the metal-organic framework used in the present invention, and specifically a metal-organic framework of imidazole and a zinc atom (average particle diameter: 0.2 μm). This metal-organic framework was obtained by mixing 60 mL of N,N-dimethylformamide solution of 0.15 M imidazole and 20 mL of N,N-dimethylformamide solution of 0.15 M zinc nitrate, and heating and depositing the mixture at 100° C. for 24 hours in a stainless steel jacket.

The “ZIF-7” used in Example 2 is a substance included in the category of the metal-organic framework used in the present invention, and specifically a metal-organic framework of benzimidazole and a zinc atom (average particle diameter: 0.2 μm). This metal-organic framework was obtained by mixing 60 mL of N,N-dimethylformamide solution of 0.2 M benzimidazole and 20 mL of N,N-dimethylformamide solution of 0.2 M zinc nitrate, and heating and depositing the mixture at 140° C. for 24 hours in a stainless steel jacket.

The “ZIF-8” used in Example 3 is a substance included in the category of the metal-organic framework used in the present invention, and specifically a metal-organic framework of 2-methylimidazole and a zinc atom (average particle diameter: 0.2 μm). This metal-organic framework was obtained by mixing 60 mL of an aqueous solution of 0.2 M 2-methylimidazole and 20 mL of an aqueous solution of 0.2 M zinc nitrate, and heating and precipitating the mixture at room temperature for 24 hours.

The “ZIF-9” used in Example 4 is a substance included in the category of the metal-organic framework used in the present invention, and specifically a metal-organic framework of benzimidazole and a cobalt atom (average particle diameter: 0.2 μm). This metal-organic framework was obtained by mixing 60 mL of N,N-dimethylformamide solution of 0.2 M benzimidazole and 20 mL of N,N-dimethylformamide solution of 0.2 M cobalt nitrate, and heating and depositing the mixture at 140° C. for 24 hours in a stainless steel jacket.

The “ZIF-75” used in Example 5 is a substance included in the category of the metal-organic framework used in the present invention, and specifically a metal-organic framework of 2-nitroimidazole, 2-methylbenzimidazole, and a cobalt atom (average particle diameter: 0.2 μm). This metal-organic framework was obtained by mixing 60 mL N,N-dimethylformamide solution of 0.15 M nitroimidazole and 0.15 M 2-methylbenzimidazole and 20 mL of N,N-dimethylformamide solution of 0.2 M cobalt nitrate, and heating and depositing the mixture at 85° C. for 24 hours in a stainless steel jacket.

The “Fe(Im)₂” used in Example 6 is a substance included in the category of the metal-organic framework used in the present invention, and specifically a metal-organic framework of imidazole and a zinc atom (average particle diameter: 0.2 μm). This metal-organic framework was obtained by mixing 60 mL of N,N-dimethylformamide solution of 0.2 M imidazole and 20 mL of N,N-dimethylformamide solution of 0.2 M iron nitrate, and heating and depositing the mixture at 140° C. for 24 hours in a stainless steel jacket.

The “Cu-BTC” used in Comparative Example 1 is a substance included in the category of so-called metal-organic framework, but not included in the category of the metal-organic framework used in the present invention (specifically, a metal-organic framework of 1,3,5-benzenetricarboxylic acid and a copper atom) (average particle diameter: 0.2 μm), and has a carboxyl group (water-absorbent group) as a substituent.

The “CPO-27-Zn” used in Comparative Example 2 is a substance included in the category of so-called metal-organic framework, but not included in the category of the metal-organic framework used in the present invention (specifically, a metal-organic framework of 2,5-dioxide-1,4-benzene-dicarboxylate and a zinc atom) (average particle diameter: 0.2 μm), and has a carboxylate group (water-absorbent group) as a substituent.

The “Zn (Adenine)” used in Comparative Example 3 is a substance included in the category of so-called metal-organic framework, but not included in the category of the metal-organic framework used in the present invention (specifically, a metal-organic structure of a purine-based molecule and a zinc atom represented by the following general formula) (average particle diameter: 0.2 μm), and has an amino group (water-absorbent group) as a substituent.

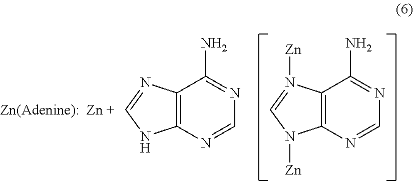

The “4A type zeolite” used in Comparative Example 4 is a substance not included in the category of a so-called metal-organic framework (average particle diameter: 0.2 μm), and specifically, is an inorganic framework containing no organic molecules.

[Evaluation]

(CO₂ Suction Amount)

The CO₂ suction amount of the non-aqueous electrolytic solution was measured according to the method illustrated in FIG. 4. The details are as follows.

(1) A measurement exterior body 51 was prepared. The exterior body 51 was obtained by heat-sealing three outer peripheral edge portions and a central portion 60 of two rectangular laminate films in plan view. By forming the seal portion of the central portion 60, a gas suction chamber 51a and a gas injection chamber 51b are provided. In forming the seal portion of the central portion 60, a non-seal portion 61 for moving CO₂ gas as described later was provided. The gas injection chamber 51b is provided with an injection port 52 for injecting gas.

(2) The exterior body 51 was folded back at the heat-sealed portion of the central portion 60, and 2 mL of the non-aqueous electrolytic solution obtained in each Example/Comparative Example was injected from a cavity of the gas suction chamber 51a. In the lower portions of the gas suction chamber 51a and the gas injection chamber 51b, the mutual movement of the contents of both chambers is restricted by clips 53.

(3) The cavity of the gas suction chamber 51a was heat-sealed and weighed. A weight Ws of only a test specimen was calculated from the weight including the clip 53, the gas injection port 52, and the test specimen (that is, the electrolytic solution-sealed exterior body) and the weight including the clip 53 and the gas injection port 52.

(4) CO₂ gas (1.5 mL) was injected into the gas injection chamber 51b through the gas injection port 52.

(5) A vicinity portion 55 of the gas injection port 52 in the gas injection chamber 51b was heat-sealed to prevent gas leakage.

(6) The volume (V₁) of the test specimen with the clip 53 was measured according to the Archimedes principle.

(7) The clip 53 was removed, the CO₂ gas was moved from the gas injection chamber 51b to the gas suction chamber 51a, and the CO₂ gas was sufficiently adsorbed.

(8) After the suction of CO₂ gas, the volume (V₂) of the test specimen with the clip 53 was measured according to the Archimedes principle.

From the values measured by the above method, the gas suction amount was calculated according to the following equation, and evaluated according to the following criteria.

Gas suction amount (mL/g)=(V₁− V₂)/Ws

⊙: 40 mL/g ≤gas suction amount (best);

∘: 30 mL/g ≤gas suction amount <40 mL/g (excellent);

Δ: 10 mL/g ≤gas suction amount <30 mL/g (no practical problem); and

x: Gas suction amount <10 mL/g (practical problem).

(H₂O Suction Amount)

The H₂O suction amount was measured by BELSORP MAXII (available from MicrotracBEL Corp.) using the sample used in each Example/Comparative Example, and evaluated according to the following criteria.

⊙: H₂O suction amount<1.0 mL/g (best);

∘: 1.0 mL/g<H₂O suction amount<10.0 mL/g (excellent);

Δ: 10.0 mL/g<H₂O suction amount<15.0 mL/g (no practical problem); and

x: 15.0 mL/g_{<}H₂O suction amount (practical problem).

(Decomposition Rate of Lithium Salt)

The non-aqueous electrolytic solution obtained in each Example/Comparative Example was stored in an 80° C. thermostatic bath in a dry room for 3 days.

Three days after the storage, the non-aqueous electrolytic solution (that is, the dispersion) was subjected to centrifugation (15,000 rpm, 30 minutes) to separate the sample (for example, zeolite or MOF) from a supernatant, and the supernatant solution was collected to obtain a sample solution.

The FT-IR spectrum of this sample solution was measured, and the decomposition rate of the lithium salt was obtained from the peak intensity ratio between the peak at 840 cm⁻¹, which is a peak of a P-F bond of LiPF₆, and a peak at 1070 cm⁻¹, which is a peak of an ether bond of the carbonate. The decomposition rate was evaluated according to the following criteria.

⊙: decomposition rate=0% (best);

∘: 0%<decomposition rate<1.0% (excellent);

Δ: 1.0%<decomposition rate<2.0% (no practical problem); and

x: 2.0%<decomposition rate (there is a problem in practical use).

TABLE 1

| | | | | | | | H₂O | |
|---|---|---|---|---|---|---|---|---|
| | | | | | | CO₂ | suction | |
| | | | | | | suction | amount | |
| Examples/ | Samples | | | | Pore | amount | (ml/g) | Decomposition |
| Comparative | | Metal | | Composition | diameter | (ml/g) | 1% RH | rate of |
| Examples | Types | atom | Organic molecule | formula | (Å) | 1bar 298K | 298K | lithium salt |
| Example 1 | ZIF-4 | Zn | Imidazole (Im) | Zn (Im)₂ | 2.1 | 49.28⊙ | 0.06⊙ | 0%⊙ |
| Example 2 | ZIF-7 | Zn | Benzimidazole (bIm) | Zn (bIm)₂ | 4.31 | 35.62◯ | 0.94⊙ | 0%⊙ |
| Example 3 | ZIF-8 | Zn | 2-methylimidazole (mIm) | Zn (mIm)₂ | 11.6 | 14.93Δ | 0.31⊙ | 0%⊙ |
| Example 4 | ZIF-9 | Co | Benzimidazole (bIm) | Co (bIm)₂ | 4.31 | * | * | * |
| Example 5 | ZIF-75 | Co | 2-methylbenzimidazole (mIm) | Co (mIm) (nIm) | 2.62 | * | * | * |
| | | | 2-nitroimidazole (nIm) | | | | | |
| Example 6 | Fe (Im) ₂ | Fe | Imidazole (Im) | Fe (Im) ₂ | 2.8 | * | * | * |
| Comparative | Cu•BTC | Cu | 1,3,5-benzenetricarboxylic | Cu₃ (BTC)₂ | 16.6 | 107.71⊙ | 43.88X | 5%X |
| Example 1 | | | acid (BTC) | | | | | |
| Comparative | CPO-27-Zn | Zn | 2,5-dioxido-1,4-benzene- | Zn₂ (dhtp) | 13.9 | 9.30X | 148.03X | 6%X |
| Example 2 | | | decarboxylate (dhtp) | | | | | |
| Comparative | Zn | Zn | Adenine | Zn (Adenine) 2 | Unclear | 22.869Δ | 17.452X | 3%X |
| Example 3 | (Adenine) | | | | | | | |
| Comparative | 4A type | — | | Na₁₂ | 5.1 | 89.4⊙ | 168.19X | 19%X |
| Example 4 | zeolite | | | [(AlO₂) ₁₂ (SiO₂) ₁₂] | | | | |

*: Measurement was not performed

When a secondary battery was produced using the non-aqueous electrolytic solution obtained in each Example/Comparative Example, the secondary battery had the original function of the secondary battery.

When an electric double-layer capacitor was produced using the non-aqueous electrolytic solution obtained in each Example/Comparative Example, the electric double-layer capacitor had the original function of the electric double-layer capacitor.

The electrochemical device of the present invention can be used in various fields where battery use and electric storage are assumed. Although it is merely an example, the electrochemical device according to the present invention, in particular, the secondary battery and the electric double-layer capacitor can be used in the field of electronics mounting. The electrochemical device according to an embodiment of the present invention can also be used in the fields of electricity, information, and communication in which mobile equipment, and the like are used (for example, electric and electronic equipment fields or mobile equipment fields including mobile phones, smartphones, notebook computers and digital cameras, activity meters, arm computers, electronic papers, and small electronic machines such as wearable devices, RFID tags, card type electronic money, and smartwatches), home and small industrial applications (for example, the fields of electric tools, golf carts, and home, nursing, and industrial robots), large industrial applications (for example, fields of forklift, elevator, and harbor crane), transportation system fields (field of, for example, hybrid automobiles, electric automobiles, buses, trains, power-assisted bicycles, and electric two-wheeled vehicles), power system applications (for example, fields such as various types of power generation, road conditioners, smart grids, and household power storage systems), medical applications (medical equipment fields such as earphone hearing aids), pharmaceutical applications (fields such as dosage management systems), IoT fields, space and deep sea applications (for example, fields such as a space probe and a research submarine), and the like.

### DESCRIPTION OF REFERENCE SYMBOLS

1: Non-aqueous electrolytic solution
2: Positive electrode
3: Negative electrode
4: Separator
5: Exterior body
10: Secondary battery
20: Electric double-layer capacitor
21: Non-aqueous electrolytic solution
22: Positive electrode
23: Negative electrode
24: Separator
27: Exterior body

1. An electrochemical device comprising a non-aqueous electrolytic solution, wherein the non-aqueous electrolytic solution contains a metal-organic framework containing: an azole-based organic molecule optionally having a hydrophobic group, and a metal atom.

2. The electrochemical device according to claim 1, wherein the azole-based organic molecule is an organic molecule selected from the group consisting of imidazole, benzimidazole, triazole, and purine.

3. The electrochemical device according to claim 1, wherein the azole-based organic molecule is an imidazole, and the metal atom is one or more selected from the group consisting of a zinc atom and an iron atom.

4. The electrochemical device according to claim 1, wherein the azole-based organic molecule is a benzimidazole, and the metal atom is one or more selected from the group consisting of a zinc atom and a cobalt atom.

5. The electrochemical device according to claim 1, wherein the azole-based organic molecule is a triazole, and the metal atom is one or more selected from the group consisting of a zinc atom and a cobalt atom.

6. The electrochemical device according to claim 1, wherein the azole-based organic molecule is a purine, and the metal atom is one or more selected from the group consisting of a zinc atom and a cobalt atom.

7. The electrochemical device according to claim 1, wherein the azole-based organic molecule is a combination of an imidazole and a benzimidazole, and the metal atom is a cobalt atom.

8. The electrochemical device according to claim 2, wherein the azole-based organic molecule has the hydrophobic group, and the hydrophobic group is a substituent selected from the group consisting of an alkyl group, a halogen atom, a nitro group, a phenyl group, a pyridyl group, and a cyano group.

9. The electrochemical device according to claim 8, wherein the metal atom is selected from the group consisting of a zinc atom, a cobalt atom, an iron atom, a praseodymium atom, a cadmium atom, a mercury atom, a copper atom, an indium atom, a manganese atom, a lithium atom, and a boron atom.

10. The electrochemical device according to claim 1, wherein the azole-based organic molecule has the hydrophobic group, and the hydrophobic group is a substituent selected from the group consisting of an alkyl group, a halogen atom, a nitro group, a phenyl group, a pyridyl group, and a cyano group.

11. The electrochemical device according to claim 1, wherein the metal atom is selected from the group consisting of a zinc atom, a cobalt atom, an iron atom, a praseodymium atom, a cadmium atom, a mercury atom, a copper atom, an indium atom, a manganese atom, a lithium atom, and a boron atom.

12. The electrochemical device according to claim 1, wherein the azole-based organic molecule is an imidazole-based molecule represented by at least one of the following: 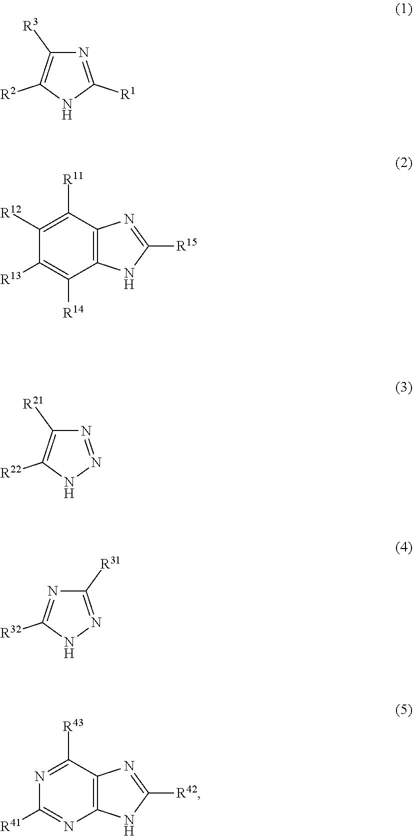
wherein, R¹ to R³, R¹¹ to R¹⁵, R²¹, R²², R³¹, R³², and R⁴¹ to R⁴³ are each independently a hydrogen atom, an alkyl group, a halogen atom, a nitro group, a phenyl group, a pyridyl group, or a cyano group.

13. The electrochemical device according to claim 1, wherein the metal-organic framework has a pore diameter of 1 Å to 50 Å.

14. The electrochemical device according to claim 1, wherein the metal-organic framework has a pore diameter of 1 Å to 15 Å.

15. The electrochemical device according to claim 1, wherein the metal-organic framework has a pore diameter of 2 Å to 5 Å.

16. The electrochemical device according to claim 1, wherein the metal-organic framework has a pore diameter of 2 Å to 3 Å.

17. The electrochemical device according to claim 1, wherein the non-aqueous electrolytic solution contains the metal-organic framework in an amount of 0.1 wt. % to 50 wt. % with respect to a total amount of the non-aqueous electrolytic solution.

18. The electrochemical device according to claim 1, wherein the non-aqueous electrolytic solution further contains an organic solvent and an electrolyte salt.

19. The electrochemical device according to claim 1, wherein the electrochemical device is a lithium ion secondary battery or an electric double-layer capacitor.

20. The electrochemical device according to claim 1,
wherein the electrochemical device is a lithium ion secondary battery,
the lithium ion secondary battery further contains a positive electrode and a negative electrode, and
the positive electrode and the negative electrode have a layer capable of occluding and releasing a lithium ion.
